(19) United States
(12) Patent Application Publication
(10) Pub. No.: US 2004/0122419 A1
(43) Pub. Date: Jun. 24, 2004

(54) Medical device recognition system with write-back feature

(75) Inventor: Wolfgang Neuberger, F.T. Labuan (MY)

Correspondence Address: BOLESH J. SKUTNIK PhD, JD, 515 Shaker Road, East Longmeadow, MA, 01028, US

(73) Assignee: CeramOptec Industries, Inc.

(21) Appl. No.: 10/322,180

(22) Filed: Dec. 18, 2002

(51) Int. Cl. A61B 18/20

(52) U.S. Cl. 606/10; 606/13; 606/16

(57) ABSTRACT

A medical radiation treatment system is disclosed for identifying and monitoring the use of disposable or reusable optical fibers or other optical accessories. The treatment system comprises a radiation source unit connected to a recognition/control unit, and a medical radiation delivery system connected to an identification/recordation unit. This forms a read-write system to ensure that radiation parameters conform to delivery device characteristics, and to prevent the use of overused or incompatible delivery devices. This is achieved by reading previously encoded information in the identification/recordation unit that provides all usage history, including number of uses or total duration of use. The encoded information is updated after use, and preferably frequently or continuously during use, to guarantee a complete usage history even after an incomplete treatment. Additionally, the treatment system can differentiate between completed treatments, incomplete treatments and calibration, to retain an accurate record of all the stresses on a fiber. A transponder sends information to the source unit, and also receives and writes information to a non-volatile memory chip or other storage means. The identification/recordation unit, including the transponder and memory, is powered by sending/receiving means in the recognition/control unit without physical, optical or electrical connection between the source unit and delivery system.

### BACKGROUND OF THE INVENTION

1. Field of the Invention

The present invention relates to medical laser delivery systems with recognition subsystems to prevent incompatible or overused optical fiber probes from being coupled with a laser.

2. Information Disclosure Statement

A number of distinct systems are known in the prior art for increasing the safety of laser systems by restricting the type of optical fiber that can be coupled with a given laser. For medical laser delivery systems and other laser systems, various concepts have been patented such as bar codes and delivery-type-dependent resistors in the devices or in connectors. These systems have been used both for restricting the allowable fibers and for automatically adjusting the parameters of the laser beam to conform with the fiber characteristics. The prior art has also disclosed means of limiting the use of a laser delivery system such as an optical fiber to a predetermined number of cases so as to attempt to ensure its safety and viability.

For medical laser applications, attached fiber optic equipment must be properly matched with the laser source. Characteristics such as maximum power, pulse frequency, fiber type and diameter, and optimum wavelength range should be matched between the laser and the fiber to avoid unnecessary damage to the fiber or, more importantly, the patient, and to maximize therapeutic effect. Also, it is important that disposable equipment not be used more than once and that equipment such as optical fibers be limited in their use so that they can be discarded before the level of degradation of the fiber, from repeated sterilization as well as irradiation, is severe enough to compromise treatment quality. It is further desirable that a device be able to guarantee only an approved number of uses, and is also useful to have a device that limits the use of a fiber depending on the amount of delivered energy used.

U.S. Pat. No. 5,681,307 by McMahan et al describes a system for communicating information from a detachable fiber optic appliance to a base unit so that the base unit can calibrate itself to match the appliance. This invention recognizes that laser sources can be suitable for a number of different fiber optic appliances, but that each requires calibration by the user, which can be inefficient and prone to error. This invention works on the premise that fiber optic appliances used are disposed of after each use to ensure sterilization and avoid using appliances that have been worn out. Because of this, McMahan allows only for reading information on the disposable fiber, so does not allow for the possibility of encoding information from the laser onto the appliance.

A variation is described in U.S. Pat. No. 6,068,627 by Orszulak et al. in which a connection means for coupling an energy source and a medical instrument for use in electrosurgery is provided to ensure that only the proper instruments are used with the source. Unlike the above patent, this invention seeks to exclude improper instruments rather than conform itself to the treatment parameters of a range of instruments. Identification is accomplished through the use of infrared light, which is used to communicate a unique code identification between the instrument and source. This invention is limited to identification, and further has no means for writing or storing use information in either the energy source or the instrument.

U.S. Pat. No. 5,400,267 describes a non-volatile memory device within instruments for tracking the usage of limited-use instruments. This system would prevent an instrument from being used if the memory recorded a greater number of uses than a preset limit. Also, the memory can be used to automatically set operational parameters used by the supply/control/measuring apparatus or prevent non-compatible apparatus from being used with the instrument. The device stores the use information and can deliver it to any appliance used with the instrument. This invention discusses reusable electrical medical instruments, but does not mention or deal with optical sources such as lasers. Also, this invention requires an electrical connection. The control device is attached to the power supply and will turn off the system. U.S. Pat. No. 6,068,627, described above, makes the following comment about this patent: “A problem arises when the memory is located external to the power supply requiring hardwire connections. The communicated data transmission from the memory to the control may have an error due to radiated emissions from radio frequency energy wires located closely when delivered by the electrosurgical generator during surgery. Radio frequency exposure will interfere with the identification information being transmitted so it becomes difficult to determine that the correct medical instrument is attached to the power source.”

U.S. Pat. No. 6,308,089 describes an electrical medical appliance monitoring device connected to a medical probe. The monitoring device is an integrated device consisting of a controlling means, memory storage means, and a display. The controlling means is preferably a microprocessor.

The probe consists of a sensor and a memory storage component for storing both use and recognition information. The memory storage primarily serves to store use value, or information on the number and/or duration of use. Other parameters and sets of data can also be stored.

An initial step consists of a query by the monitoring device to determine that the probe is a proper probe and that it is operational. If there is no verification from the probe, the monitoring device display indicates such and the monitoring device prevents the probe from being used. Also provided are serial number and encryption information for added security. After the probe is properly identified, the monitoring device stores the use value from the probes memory storage. This value is compared to a maximum use value, which if exceeded, will prompt the monitoring device to prohibit use of the probe. Other information, such as the date and time of each use, can be stored to provide a fuller picture of the use history.

This system of monitoring use and preventing overuse is not contemplated for application with lasers, and thus has no means for measuring such values as would be pertinent to an optical fiber system, such as the power or energy applied through a fiber. Moreover, this invention discloses no means for distinguishing between use for a complete treatment, an incomplete treatment or use only for calibration purposes. Thus, there would be no way to accurately measure the different stresses on a fiber.

U.S. Pat. No. 4,822,997 describes an optical conductor containing non-volatile memory that stores cumulative usage values. The cumulative usage value is updated with each use and may also feature means for preventing further use of the fiber when the usage value has reached a predetermined maximum. The laser housing contains means for measuring the laser power and a shutter for regulating power. The fiber optic cable assembly, attached to the fiber and connected to the laser housing, contains non-volatile memory.

In one embodiment, power detectors provide a signal to a microprocessor that makes real time calculations of the energy supplied. That data retrieved during treatment and added to data previously taken from the non-volatile memory, provides updated cumulative usage data which is then stored in the non-volatile memory. Values for maximum usage can also be stored in the memory, which the microprocessor can compare and provide warning or control signals. Other parameters, such as the number of treatments or the number of hours used can be stored and compared by the microprocessor as described above.

This patent claims that the invention can also be utilized as an add-on to existing laser units, wherein the laser is attached via an optical conductor to a housing containing the diagnostic equipment and microprocessor, which in turn is attached to the fiber optic cable assembly. The optical parameters are transformed into electrical signals that are transferred as electrical signals to the memory. This invention is limited in that it requires an electro-optical connection before identification information can be read and utilized.

There has been no use of transponders in conjunction with calibrating or ensuring proper use of medical devices, and particularly not with optical delivery devices, although there has been use described in the context of medical equipment inventory management, as described below. It is known that, in general, identification of objects or devices can be achieved through the use of radio frequency identification (RFID) transponders, though no use of transponders has yet been made to store detailed use information and limit the use of medical devices. The patent description below is useful in illustrating that, although transponders have been contemplated in conjunction with medical equipment, there has been no contemplation or suggestion to use transponders beyond a purely organizational or inventory-tracking function.

U.S. Pat. No. 5,910,776 features an RFID system for tracking and monitoring medical equipment wherein an RFID transponder is attached to a connector, such as an electrical plug. An RFID reader is located near or in the electrical outlet and will identify the equipment. Since the location of each reader is known, the location of that equipment will be known by the information that its transponder relays to the system. This patent does have an identification aspect to it, but it is not a system for selecting or restricting equipment with the connector, and further does not provide a way to write back to the transponder device information regarding the equipment's use. It also generally requires an electrical connection between the equipment and electric plug connector.

This patent is primarily contemplated for use with general medical equipment and other equipment that can be situated in a plurality of locations and can also be coupled to a generic connecting device, such as an electrical outlet or a phone jack. The problem addressed is in identifying medical equipment location by using transponders in such a way as to overcome the limitations in the read range of the readers.

The readers in this patent can also be used to monitor and adjust the flow of current through the connectors when they are plugged in, which gives a continuous read-out of location while equipment is plugged in. Other monitoring information can include the time that the equipment was plugged in and the time the equipment was removed. Further included are RFID's that monitor the amount of time that the equipment has been used, whether equipment is functioning properly, when the equipment was used. However, this information is stored in a central processing unit, and thus is only useful within the system of connectors that is connected to the system.

The invention described in this patent is not designed for use with optical equipment and fails to include a write-back feature, where new information can be recorded onto the connector, nor is there any indication that the invention can prevent equipment from being used. This is purely a tracking and monitoring system, and does not anticipate using transponders to continuously record new information and automatically ensure that proper equipment is used or that overused equipment is not used. The benefit of using this invention with medical equipment is the often urgent need for rapid location of equipment. There is no safety or calibration aspect to this invention that would suggest the use of transponders for properly matching and calibrating medical devices. Additionally, optical equipment such as optical fibers are generally too small to be effectively used in the above system, which generally contemplates tracking large pieces that are plugged into a wall socket. Furthermore, an electrical connection is generally required in this invention.

It would be extremely useful to have a device for use in connecting optical fibers and laser sources that can conveniently and easily provide and store information to both calibrate the laser and limit the use of laser delivery devices, while being capable of maximizing the separation between the delivery device and laser prior to verification, and that can be easily incorporated with a laser source and delivery device, especially considering the size limitations on the latter.

Objectives and Brief Summary of the Invention

It is an object of the present invention to provide a medical laser system for ensuring proper use of lasers in conjunction with optical fibers.

It is another object of the present invention to ensure that the proper settings of a laser source such as calibration, parameter ranges and application software are set depending on the type of fiber used and application or medical procedure required.

It is still another object of the present invention to provide an improved system for monitoring the use of limited-use optical fibers and accessories and preventing the use of a laser or radiation delivery system with unsuitable or over-used fibers or accessories.

It is a further object of the present invention to provide a recognition and use-monitoring system that can act as an additional safety tool by preventing overuse of an optical delivery device without requiring a physical electric connection between the device and the laser source.

Briefly stated, the present invention discloses a medical radiation treatment system for identifying and monitoring the use of disposable or reusable optical fibers or other optical accessories. The treatment system comprises a radiation source unit connected to a recognition/control unit, and a medical radiation delivery system connected to a second identification/recordation unit. This forms a read-write system to ensure that radiation parameters conform to delivery device characteristics, and to prevent the use of overused or incompatible delivery devices. This is achieved by reading previously encoded information in the identification/recordation unit that provides all usage history, including number of uses or total duration of use. The encoded information is updated after use, and preferably frequently or continuously during use, to guarantee a complete usage history even after an incomplete treatment. Additionally, the treatment system can differentiate between completed treatments, incomplete treatments and calibration, to retain an accurate record of all the stresses on a fiber. A transponder sends information to the source unit, and also receives and writes information to a non-volatile memory. The identification/recordation unit, including the transponder and memory, is powered by sending/receiving means in the recognition/control unit without physical, optical or electrical connection between the source unit and delivery system. This feature of the present invention increases the safety of the system by ensuring that there is no possibility of unintended transmission through the delivery device prior to proper identification and calibration.

The above, and other objects, features and advantages of the present invention will become apparent from the following description read in conjunction with the accompanying drawings, in which like reference numbers in different drawings designate the same elements.

### BRIEF DESCRIPTION OF FIGURES

FIG. 1—Side view of connector system.

FIG. 2—Side views of delivery device unit.

FIG. 3—View of ending unit attached to laser source.

FIG. 4—Schematic of a preferred embodiment of the present invention.

### DETAILED DESCRIPTION OF PREFERRED EMBODIMENTS

The present invention is a safer and more versatile connection system for restricting the use of laser delivery accessories than has been provided in the prior art. The disclosed medical radiation treatment system features a radiation source and a medical radiation delivery system. A recognition/control unit and an identification/recordation unit are attached to the radiation source and to the delivery system, respectively, to automatically prevent the use of incompatible or overused laser delivery devices from being inadvertently utilized in a medical or other treatment. In this way, the treatment system easily helps to ensure increased safety and treatment effectiveness. The use of a transponder-receiver system allows the connector to verify the delivery device prior to connection and provides power to the identification/recordation unit in the delivery system without the need for electrical, physical or optical connection between the radiation source unit and the delivery system. A connection means is provided to optically couple radiation from the source to the delivery system after verification of the delivery system.

The treatment system contains a read-write feature that allows the system to record and retain information regarding the amount or duration of optical delivery device use or the amount of energy that has been conducted through such a device. This is especially useful for single-use disposable devices and for reusable devices that have a maximum effective lifetime. By recording the usage of the delivery device in memory during each new treatment, the delivery device maintains a readable and continually updated usage history. This information is preferably updated frequently or continuously during treatment to ensure that accurate use values are recorded even in the event of an incomplete treatment. The treatment system can then read the encoded information about the usage of the device and compare that with a maximum use value, and thus prevent the use of the device beyond its maximum useful life.

Another benefit of the read-write feature is that it can retain information for use in automatically calibrating the radiation source so that the proper radiation parameters are used in conjunction with the delivery device. Radiation characteristics that can be stored for calibration include wavelength, power range, treatment duration, treatment modes such as continuous or pulsed, pulse duration, and pulse shape or laser spot size. Delivery device characteristics that can be stored for calibration include fiber type, diameter, maximum power levels, and application handpiece treatment modalities. Software settings can be limited depending on the desired application. The delivery device defines the software of the laser and the range of allowed treatment settings. In a preferred embodiment, the radiation delivery device is an optical fiber.

The information contained in the memory, which in a preferred embodiment is a memory chip, not only includes use and identification information, but may also include information on power limits, duration limits, permissible wavelength ranges, and other information. Such information can be used to automatically calibrate the radiation for use with that particular delivery device. During treatment, the transponder also collects information as each treatment is performed, and records such information as the number of uses, the duration of each use, and treatment parameters if needed. The identification/recordation unit can also be used for recording more detailed use information. For example, in a preferred embodiment the use information encoded to the memory by the transponder coil is distinguished between “full time use”, where a full treatment was completed, “aborted use”, “use for demonstrative purposes” or “use for calibration”. In this way, the memory chip will contain accurate information as to the exact amount of energy that has been conducted in the fiber. Alternatively, the connector can ignore incomplete treatment and calibrations so as to only count completed treatments. Situations where only completed or partially completed treatments should be counted occur when the number of sterilizations is the operative factor in determining the maximum useful life of a fiber or other instrument. In some cases, sterilization techniques will degrade a fiber more quickly than laser radiation will. Therefore, those treatments where the treatment was begun, but where the instrument did not come in contact with a patient and therefore did not need sterilization, would not be counted.

The treatment system consists of a recognition/control unit connected to a radiation source and an identification/recordation unit attached to a radiation delivery device. The radiation source may be, but is not limited to, a laser source, at least one light-emitting diode (LED), at least one superluminescent diode, or a high power lamp. The recognition/control unit can be built into the radiation source itself or otherwise coupled to the source. In one preferred embodiment, the recognition/control unit is incorporated into a connector that is optically coupled to the radiation source so that the recognition/control unit can be interchanged with other radiation or laser sources. The recognition/control unit contains a sending/receiving means, which is preferably in the form of a coil or electromagnet, electrically connected to the radiation unit power source or, alternatively, connected to an outside power source so as to maintain its interchangeability. The sending/receiving means, when connected to an electric current, emits a magnetic field that inductively powers the delivery device unit. The recognition/control unit also contains an antenna for receiving radio frequency signals emitted by the identification/recordation unit. This antenna can be incorporated into the sending/receiving means or the sending/receiving means itself can serve as the antenna. These signals contain pertinent information about the delivery device such as identification information and use history. This information is then passed on to a control device such as a microprocessor, which processes the information and then determines whether to allow an optical connection between the radiation source unit and the delivery device.

The required information, which is used to determine whether a delivery device is suitable for use with a given radiation source, is housed within the identification/recordation unit. The identification/recordation unit is attached to the optical delivery device and can be read by any suitably equipped radiation source or by the recognition/control unit connected to any source. The identification/recordation unit contains, in a preferred embodiment, a non-volatile memory for storing identification and use information that is sent to the control device to determine whether the delivery device is proper for the radiation source. A radio frequency identification (RFID) transponder is also included in the identification/recordation unit, which both records use information on the non-volatile memory and sends information to the recognition/control unit.

The safety and efficacy benefits described above are achieved by reading the encoded information providing the proper use of the radiation delivery device and containing all the usage history. After the completion of each new procedure, information stored in the memory chip is updated, so as to make its history complete. Updating can also be done at the start and/or during the course of a medical treatment to make sure that system failures or faulty human interaction cannot lead to an incomplete history record on the device. A removable blockage, which would serve to permanently preserve the information and prevent further writing to the chip, may further insure that the device history is complete. This blockage would be useful as a further check against multiple uses of single-use devices, in that it could be removed after a single treatment. Likewise, for limited use devices, a user can remove the blockage after the control device notifies the user that the usage limit has been reached. In this way, a complete and accurate history is preserved in that the risk of inadvertently adding new usage material is eliminated. The blockage may also visually identify the device as having been completely used up, preventing inadvertent wrong inventory counts.

Unlike the prior art, the present invention does not require a physical, electrical or optical connection between the radiation source and the delivery device in order to identify the delivery device. The present invention accomplishes this in two ways. First, the present invention utilizes a radio frequency identification (RFID) transponder to communicate with the connector or the radiation source. The transponder transmits information to the recognition/control unit or source through radio waves, including identification and usage history information. Another unique and novel aspect of the present invention is that the identification/recordation unit is powered inductively by the sending/receiving means, instead of requiring a direct electrical connection as in the prior art.

The non-volatile memory in the identification/recordation unit is also powered purely by induction. This is a significant advantage over the prior art, in that the system described can determine whether the radiation delivery device is suitable for the radiation source before there is an actual physical connection between the radiation source and the delivery device. In the prior art, a connection was required in order to electrically power the memory or information transmitting devices in the delivery devices. This could leave open the possibility of inadvertent transmission of radiation into the delivery device due to human error or mechanical malfunction. It can also make it more difficult for a user to force transmission due to a lack of physical connection. This risk is eliminated with the present invention.

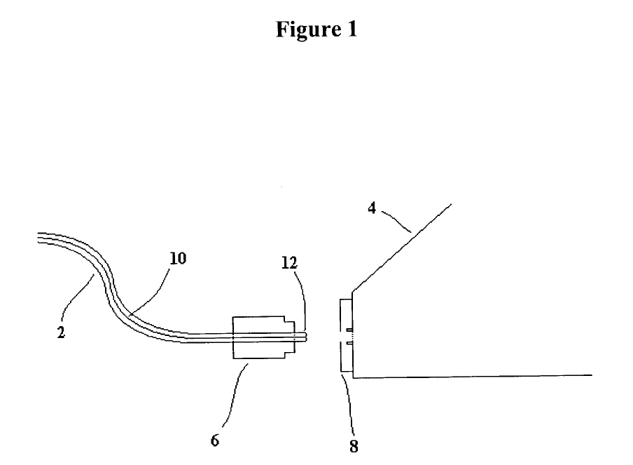

A preferred embodiment of the present invention is more fully illustrated by the following figures. FIG. 1 illustrates a side view of the entire connector setup. Delivery device 2 containing optical fiber 10 is coupled with laser source 4 for the delivery of treatment radiation to a treatment area. Previously existing plug 12 will remain the means by which the delivery device is optically connected to the laser beam. Identification/recordation unit 6 is attached to delivery device 2 near its proximal end. The proximal end of delivery device 2 then fits into laser source unit 8 via plug 12.

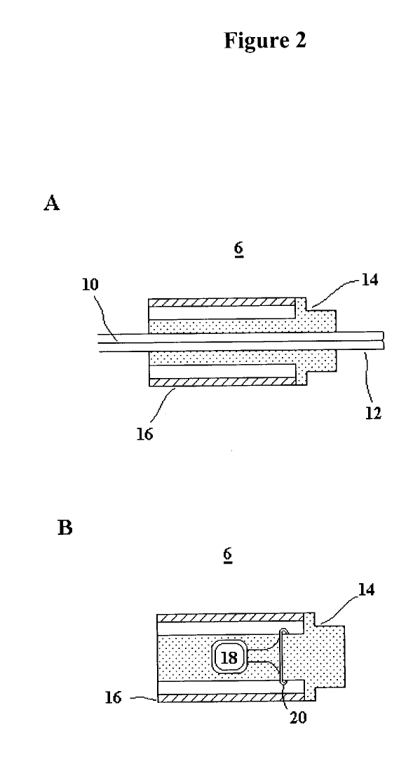

FIGS. 2a and 2b provide a more detailed picture of identification/recordation unit 6. Identification/recordation unit 6 consists of two cylinders, inner cylinder 14 and outer cylinder 16, which surround device 2 proximal to the laser source. Identification/recordation unit 6 is positioned along device 2 so that plug 12 is exposed and can still be coupled with coupling means already existing on laser source 4. Attached to inner cylinder 14 and protected by outer cylinder 16 is transponder 18, into which a memory chip is incorporated, and transponder coil 20.

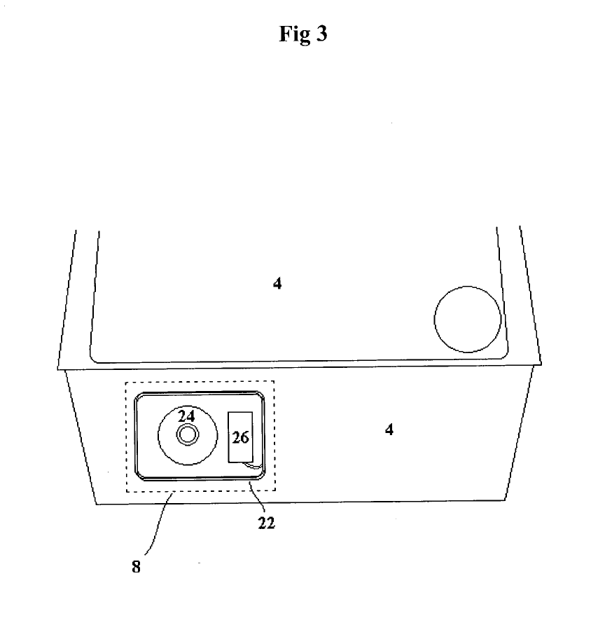

Finally, FIG. 3 provides an illustration of the treatment system's recognition/control unit 8. Recognition/control unit 8 comprises sending/receiving coil 22, which surrounds fiber socket 24, and card module 26, both of which are fixed within the laser source 4. Card module 26 contains suitable electronics and is connected to a control means such as a microprocessor. Because, as in this embodiment, recognition/control unit 8 is fastened to the exterior of laser source 4, recognition/control unit 8 can easily be removed for replacement or attachment to a different laser source. Alternatively, recognition/control unit 8 may be incorporated within source 4 or manufactured with source 4.

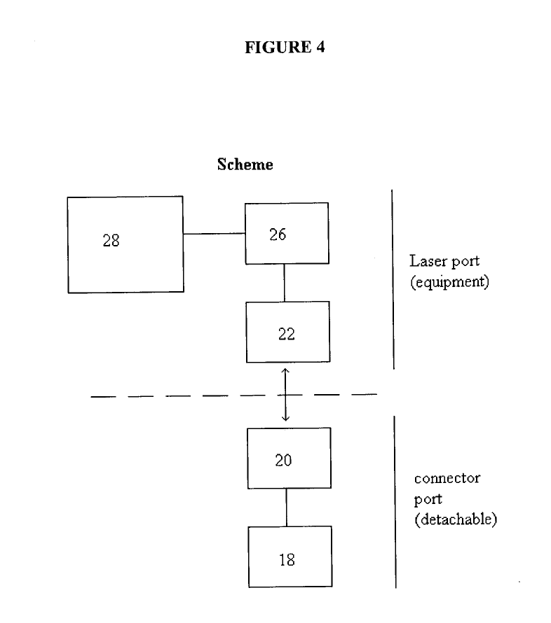

A preferred setup is illustrated by the schematic in FIG. 4. Sending/receiving coil 22, when current is applied, produces a magnetic field, which in turn induces a current in transponder coil 20. Transponder 18, now powered inductively by the laser source unit, transmits the required identification, calibration, or use information to card module 26, which in turn relays the information to control device 28. Control device 28 is preferably a microprocessor incorporated into the laser source, or a computer connected to the source.

As an illustration, the following description details how the present invention would work in practice.

1) Prior to the first treatment, a new fiber is fitted with identification/recordation unit 6. This can be permanently fitted without modification to the original connector. Also, recognition/control unit 8, if interchangeable, is fitted to the laser source.

2) Initially, basic information about the fiber is recorded onto the memory chip. Such information includes fiber type and material makeup, fiber diameter, usage restrictions, and restrictions, if any, on the type of treatments allowable. Also entered initially are laser wavelength and power restrictions.

3) The proximal end of the fiber is connected to or placed near recognition/control unit 8, which is connected to a control means such as a microprocessor. An input means is also provided, and in a preferred embodiment, the control means is a computer. Transponder 18 is inductively powered by recognition/control unit 8, which then sends the initial information to recognition/control unit 8. This information is sent to the microprocessor or computer, which determines whether the fiber is suitable for use with the laser source. If not, the microprocessor will not allow the laser to be activated while the fiber is connected or nearby. If the fiber is suitable, further calibration and use will be allowed.

4) Record-keeping information such as the date, laser type and treatment type is sent to the chip to be stored as part of a permanent record.

5) Treatment is commenced. Use information such as power and pulse rate is frequently or continuously sent to the identification/recordation unit during treatment.

6) Upon completion of treatment, final usage information is sent to identification/recordation unit and usage history is permanently updated.

7) In subsequent treatments, the updated usage information is compared to set limits previously entered to the identification/recordation unit. This information, along with fiber type and restrictions, is assessed by the microprocessor or computer to determine whether the fiber can be used.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A medical radiation treatment system with recognition and write-back capability for improved safety and efficiency, having a radiation source and a medical radiation delivery system, comprising:
a recognition/control unit, connected to said radiation source, comprising means for sending and receiving radio frequency signals, and means to control use of said medical radiation delivery system with said radiation source;
an identification/recordation unit attached to said medical radiation delivery system comprising a transponder and a read/write, nonvolatile memory;
wherein said memory stores information about said delivery system to be used by said control means to prevent improper use of said delivery system;
wherein said transponder can transmit information from said memory to said control means, and can encode new information onto said memory as said optical delivery device is used with said radiation source; and
wherein said transponder is powered by said sending/receiving means in said recognition/control unit without the need for physical, optical or electrical contact.

2. The medical radiation treatment system according to claim 1, wherein said radiation source is selected from a group consisting of a laser source, at least one LED, at least one superluminescent diode, and a high power lamp.

3. The medical radiation treatment system according to claim 1, wherein said optical delivery device is an optical fiber.

4. The medical radiation treatment system according to claim 1, wherein said information comprises identification information, use information and calibration information.

5. The medical radiation treatment system according to claim 4, wherein said identification information comprises type, material makeup and diameter of said fiber

6. The medical radiation treatment system according to claim 4, wherein said use information comprises duration of use, number of treatments, parameters of past treatments, maximum number of uses, and maximum duration of use.

7. The medical radiation treatment system according to claim 4, wherein said use information comprises classifications selected from a group consisting of full-time use, aborted use, demonstrative use and use for calibration.

8. The medical radiation treatment system according to claim 4, wherein said control means allows commencement of treatment only if:
identification information sent from identification/recordation unit is compatible with laser source; and
amount of use of said delivery device is within maximum use parameters sent from identification unit.

9. The medical radiation treatment system according to claim 1, wherein said identification/recordation unit further comprises a removable blockage, wherein upon removal of said blockage, further information cannot be recorded onto said memory chip.

10. The medical radiation treatment system according to claim 1, wherein said recognition/control unit is permanently connected to said laser source.

11. The medical radiation treatment system according to claim 1, wherein said recognition/control unit is temporarily connected to said laser source.

12. The medical radiation treatment system according to claim 1, wherein said control means is a microprocessor and software designed to control output from said radiation source.

13. The medical radiation treatment system according to claim 1, wherein said means for sending and receiving radio frequency signals is selected from the group consisting of an antenna and said sending/receiving coil.

14. A method for identifying and calibrating a medical radiation delivery device, comprising the steps of:
a. inputting information into a memory in an identification/recordation unit attached to said delivery device, wherein said information comprises identification and maximum use information;
b. placing said delivery device in proximity to a radiation source without requiring a physical, electrical or optical connection;
c. allowing a recognition/control unit to determine whether said delivery device can properly be used with said radiation source, and whether said delivery device has been overused;
d. if said delivery device may be used with said source based on previously entered parameters into said recognition/control unit, optically connecting said delivery device to said radiation source.

15. A method for identifying and calibrating a medical radiation delivery device according to claim 14, comprising the further steps of:
c1. automatically transferring calibration information from said identification/recordation unit to said recognition/control unit;
e. allowing said recognition/control unit to modify said source to emit the proper radiation characteristics for said delivery device.

16. A method for identifying and calibrating a medical radiation delivery device according to claim 14, comprising the further step of:
e. applying radiation to a treatment area
f. recording use information during treatment to update said information contained in said memory located in said identification/recordation unit.

17. A method for preventing improper use of a medical radiation delivery system using the medical radiation treatment system of claim 1, comprising the steps of:
a. incorporating said delivery system and said radiation source to said medical radiation treatment system;
b. reading information about said delivery system from said identification/recordation unit on said delivery system;
c. determining whether said delivery system may be properly used with said radiation source; and
d. performing a medical treatment with said delivery device and said radiation source if said treatment system determines that said delivery system may be safely used.

18. A method for preventing improper use of a medical radiation delivery device according to claim 17, wherein said improper use is defined as use of said medical radiation delivery device after it has reached its “safety limit”, wherein further said “safety limit” is a preselected maximum amount of use of said delivery device.

19. A method for preventing improper use of a medical radiation delivery device according to claim 17, wherein misuse is defined as use of said delivery system with a radiation source that emits radiation parameters that are different than the parameters preselected for said delivery device.

20. A method for monitoring the use of limited usage radiation delivery devices using the medical radiation treatment system of claim 1, comprising the steps of
a. incorporating said delivery system to said medical radiation treatment system;
b. allowing said recgnition/control unit to identify said delivery system;
c. performing a treatment with said treatment system; and
d. recording information about said treatment on said identification/recordation unit.

21. The method for monitoring the use of limited usage radiation delivery devices according to claim 20, wherein said information is selected from one or more of a group consisting of duration of use, treatment parameters and type of use.

22. The method for monitoring the use of limited usage radiation delivery devices according to claim 20, wherein said type of use is selected from a group consisting of full-time use, aborted use, demonstrative use and use for calibration.

23. The method for monitoring the use of limited usage radiation delivery devices according to claim 20, wherein said treatment parameters are selected from one or more of the group consisting of wavelength, power, pulse length and pulse rate.

24. The method for monitoring the use of limited usage radiation delivery devices according to claim 20, wherein step d is performed in a manner selected from one or more of a group consisting of continuously recording during said treatment, periodically recording during said treatment and recording after said treatment.

25. The method for monitoring the use of limited usage radiation delivery devices according to claim 20, comprising the further step of:
b1. Comparing use information located in said identification/recordation unit to preselected maximum use parameters.
b2. Preventing further use of said delivery system if said use information exceeds said parameters.
